(19) United States
(12) Patent Application Publication
(10) Pub. No.: US 2012/0276598 A1
(43) Pub. Date: Nov. 1, 2012

(54) METHOD FOR PRODUCING AN L-AMINO ACID

(76) Inventors: Ippei Inoue, Kawasaki-shi (JP); Hisashi Yasueda, Kawasaki-shi (JP)

(21) Appl. No.: 13/464,322

(22) Filed: May 4, 2012

Related U.S. Application Data
(63) Continuation of application No. PCT/JP10/69436, filed on Nov. 1, 2010.

(30) Foreign Application Priority Data
Nov. 6, 2009 (JP) 2009-255042

(51) Int. Cl. C12P 13/08; C12P 13/04

(52) U.S. Cl. 435/115; 435/106

(57) ABSTRACT

A method for efficiently producing an L-amino acid, especially L-lysine, by using a γ-proteobacterium is provided. In a method for producing an L-amino acid comprising culturing a bacterium belonging to γ-Proteobacteria and having an ability to produce an L-amino acid, for example, an Enterobacteriaceae bacterium such as Escherichia coli, in a medium containing glycerol as a carbon source to produce and accumulate the L-amino acid in the medium, and collecting the L-amino acid from the medium, a bacterium modified so that the activity of the Cnu protein is reduced is used as the bacterium.

This application is a Continuation of, and claims priority under 35 U.S.C. §120 to, International Application No. PCT/JP2010/069436, filed Nov. 1, 2010, and claims priority therethrough under 35 U.S.C. §119 to Japanese Patent Application No. 2009-255042, filed Nov. 6, 2009, the entireties of which are incorporated by reference herein. Also, the Sequence Listing filed electronically herewith is hereby incorporated by reference (File name: 2012-05-04T_US-479_Seq_List; File size: 8 KB; Date recorded: May 4, 2012).

### BACKGROUND OF THE INVENTION

1. Field of the Invention

The present invention relates to a method for producing an L-amino acid utilizing a γ-proteobacterium, and especially for producing L-lysine. L-Lysine is an industrially useful L-amino acid as an additive for animal feed, ingredient of health food, amino acid infusion solution, and so forth.

2. Brief Description of the Related Art

In Escherichia coli, the ydgT gene codes for a protein having a full length of 71 amino acid residues called Cnu (oriC-binding nucleoid-associated) protein (Kim, M. S. et al., J. Bacteriol., 187:6998-7008 (2005)), and it has been reported that the Cnu protein belongs to the Hha/YmoA family of gene regulation factors that respond to environmental changes on the basis of the amino acid sequence thereof (Paytubi, S. et al., Mol. Microbiol., 54(1):251-63 (2004)). It has also been reported that the ydgT gene product of Salmonella bacteria regulates expression of the region SPI-2 coding for genes involved in systemic infection of Salmonella bacteria in animals through interactions with the H-NS protein (Coombes, B. K. et al., Proc. Natl. Acad. Sci. U.S.A., 102:17460 (2005)).

However, involvement of the ydgT gene in metabolism, such as saccharide consumption and amino acid production, has not been understood well for any bacteria.

### SUMMARY OF THE INVENTION

### Aspects of the Invention

An aspect of the present invention is to provide a method for efficiently producing an L-amino acid, especially L-lysine, by using a γ-proteobacterium.

It has been found that L-amino acids can be efficiently produced by using a bacterium in which functional activity of the Cnu protein is reduced.

It is an aspect of the present invention to provide a method for producing an L-amino acid comprising culturing a bacterium belonging to γ-Proteobacteria and having an ability to produce the L-amino acid in a medium containing glycerol as a carbon source to produce and accumulate the L-amino acid in the medium, and collecting the L-amino acid from the medium, wherein the bacterium is a bacterium modified so that the activity of the Cnu protein is reduced as compared to a non-modified bacterium.

It is an aspect of the present invention to provide the method as described above, wherein the activity of the Cnu protein is reduced by disrupting the ydgT gene coding for the Cnu protein on the chromosome, or by reducing expression amount of the gene.

It is an aspect of the present invention to provide the method as described above, wherein the Cnu protein is a protein selected from the group consisted of:

(A) a protein having the amino acid sequence shown in SEQ ID NO: 14,
(B) a protein having the amino acid sequence shown in SEQ ID NO: 14, but including substitution, deletion, insertion, or addition of one or several amino acid residues, reduction of which activity in the bacterium results in improvement in the ability to produce the L-amino acid.

It is an aspect of the present invention to provide the method as mentioned above, wherein the ydgT gene is a DNA selected from the group consisting of:

(a) a DNA comprising the nucleotide sequence of SEQ ID NO: 13,
(b) a DNA hybridizable with a nucleotide sequence complementary to the nucleotide sequence of SEQ ID NO: 13 or a probe that can be prepared from the nucleotide sequence under stringent conditions, and coding for a protein, reduction of which activity in the bacterium results in improvement in the ability to produce the L-amino acid.

It is an aspect of the present invention to provide the method as mentioned above, wherein the L-amino acid is an aspartic acid type amino acid.

It is an aspect of the present invention to provide the method as mentioned above, wherein the L-amino acid is L-lysine.

It is an aspect of the present invention to provide the method as mentioned above, wherein the bacterium is a bacterium belonging to the family Enterobacteriaceae.

It is an aspect of the present invention to provide the method as mentioned above, wherein the bacterium is an Escherichia bacterium.

It is an aspect of the present invention to provide the method as mentioned above, wherein the bacterium is Escherichia coli.

According to the method of the present invention, L-amino acids, especially L-lysine, can be extremely efficiently produced by fermentation. Moreover, according to one embodiment of the present invention, the proliferation rate and glycerol-assimilating ability of bacteria can be improved.

### BRIEF DESCRIPTION OF THE DRAWINGS

FIG. 1 includes graphs showing (a) L-lysine concentrations, and (b) glycerol concentrations in an L-lysine production medium observed when a control strain and a ydgT gene-deficient strain of Escherichia coli are cultured in the medium.

FIG. 2 includes graphs showing (a) growth curves, and (b) glycerol concentrations in an L-lysine production medium observed when a control strain and a ydgT gene-deficient strain of Escherichia coli are cultured in the medium.

### DESCRIPTION OF THE PREFERRED EMBODIMENTS

<1> γ-Proteobacterium

The γ-proteobacterium is not particularly limited, so long as it is a microorganism that belongs to γ-Proteobacteria, and has an ability to produce an L-amino acid or can be imparted with the ability to produce L-lysine. The bacterium can be obtained by modifying a bacterium belonging to γ-Proteobacteria and having an ability to produce an L-amino acid so that the activity of the Cnu protein is reduced.

Bacteria which can be used as a parent strain to derive the bacterium, which is modified so that expression of the ydgT gene is reduced, and methods for imparting or enhancing an L-amino acid-producing ability are exemplified below. The bacterium can also be obtained by imparting an L-amino acid-producing ability to a γ-proteobacterium modified so that expression of the ydgT gene is reduced, or enhancing an L-amino acid-producing ability of a γ-proteobacterium modified so that expression of the ydgT gene is reduced.

A bacterium having an L-amino acid-producing ability (a bacterium having an ability to produce an L-amino acid) can refer to a bacterium that has an ability to produce and accumulate an L-amino acid in a medium when it is cultured in the medium. The bacterium having an L-amino acid-producing ability also refers to a bacterium having an ability to accumulate an objective L-amino acid in a medium in an amount of 0.5 g/L or more, or even 1.0 g/L or more.

Examples of the L-amino acid can include L-lysine, L-glutamic acid, L-threonine, L-valine, L-leucine, L-isoleucine, L-serine, L-aspartic acid, L-asparagine, L-glutamine, L-arginine, L-cysteine (cystine), L-methionine, L-phenylalanine, L-tryptophan, L-tyrosine, L-glycine, L-alanine, L-proline, L-ornithine, L-citrulline, and L-homoserine, and an aspartic acid type amino acid or an aromatic amino acid is a particular example. Examples of the aspartic acid type amino acid can include L-lysine, L-threonine, and L-methionine, and L-lysine is a particular example. Examples of the aromatic amino acid include L-tryptophan, L-phenylalanine, and L-tyrosine. The L-amino acid can include one kind of L-amino acid, or two or more kinds of L-amino acids.

The L-amino acid includes not only an L-amino acid in free form, but also a salt thereof, such as sulfate, hydrochloride, carbonate, ammonium salt, sodium salt, and potassium salt.

<1-1> Bacteria to be used as parent strain to derive the bacterium The bacterium of present invention can be a bacterium belonging to γ-Proteobacteria and having an L-amino acid-producing ability.

γ-Proteobacteria include bacteria belonging to the family Enterobacteriaceae, such as those of genera Escherichia, Enterobacter, Erwinia, Klebsiella, Pantoea, Photorhabdus, Providencia, Salmonella, Serratia, Shigella, Morganella, and Yersinia, and bacteria belonging to the genus Vibrio and so forth. In particular, bacteria classified into γ-Proteobacteria according to the taxonomy used in the NCBI (National Center for Biotechnology Information) database (http://www.ncbi.nlm.nih.gov/Taxonomy/Browser/wwwtax.cgi?id=91347) can be mentioned, and particular examples include an Escherichia or Pantoea bacterium belonging to the family Enterobacteriaceae, or a Vibrio bacterium.

A bacterium belonging to the genus Escherichia can mean that the bacterium is classified into the genus Escherichia according to classification known to a person skilled in the art of microbiology, although the bacterium is not particularly limited. Examples of the bacterium belonging to the genus Escherichia include, but are not limited to, Escherichia coli (E. coli).

The bacterium belonging to the genus Escherichia that can be used is not particularly limited. However, examples include, for example, the bacteria of the phyletic groups described in the work of Neidhardt et al. (Neidhardt F. C. Ed., 1996, Escherichia coli and Salmonella: Cellular and Molecular Biology/Second Edition, pp. 2477-2483, Table 1, American Society for Microbiology Press, Washington, D.C.). Specific examples include the Escherichia coli W3110 (ATCC 27325), Escherichia coli MG1655 (ATCC 47076), and the like derived from the prototype wild-type strain, K12 strain.

These strains are available from, for example, the American Type Culture Collection (Address: P.O. Box 1549, Manassas, Va. 20108, United States of America). That is, registration numbers are given to each of the strains, and the strains can be ordered by using these numbers. The registration numbers of the strains are listed in the catalogue of the American Type Culture Collection.

A bacterium belonging to the genus Pantoea can mean that the bacterium is classified into the genus Pantoea according to the classification known to a person skilled in the art of microbiology. Some species of Enterobacter agglomerans have been recently re-classified into Pantoea agglomerans, Pantoea ananatis, Pantoea stewartii, or the like, on the basis of the nucleotide sequence analysis of 16S rRNA, etc. (Int. J. Syst. Bacteriol., 43, 162-173 (1993)). Bacteria belonging to the genus Pantoea encompass such bacteria re-classified into the genus Pantoea as described above.

The Vibrio bacteria are facultative anaerobic gram-negative bacteria belonging to the family Vibrionaceae of γ-Proteobacteria, and are motile bacteria with one localized flagellum seen in fresh water or seawater. The Vibrio bacteria can be nonpathogenic Vibrio bacteria. Vibrio bacteria for which pathogenicity is not known are mentioned in Biosafety level 1 (Biosafety in Microbiological and Biomedical Laboratories (BMBL), 4th Edition, published by Office of Health and Safety (OHS)), and such Vibrio bacteria as mentioned below can be used.

Vibrio abalonicus ATCC 27390

Vibrio adaptatus ATCC 19263

Vibrio aerogenes ATCC 700797

Vibrio aestuarianus ATCC 35048

Vibrio alginolyticus ATCC 14582

Vibrio algosus ATCC 14390

Vibrio anguillarum ATCC 43305

Vibrio calviensis ATCC BAA-606

Vibrio campbellii ATCC 25920

Vibrio carchariae ATCC 35084

Vibrio coralliilyticus ATCC BAA-450

Vibrio costicola ATCC 43147

Vibrio cyclitrophicus ATCC 700982

Vibrio cyclosites ATCC 14635

Vibrio diazotrophicus ATCC 33466

Vibrio fischeri ATCC 25918

Vibrio gazogenes ATCC 29988

Vibrio halioticoli ATCC 700680

Vibrio harveyi ATCC 14126

Vibrio hispanica ATCC 51589

Vibrio ichthyoenteri ATCC 700023

Vibrio iliopiscarius ATCC 51760

Vibrio lentus ATCC BAA-539

Vibrio liquefaciens ATCC 17058

Vibrio logei ATCC 15382

Vibrio marinagilis ATCC 14398

Vibrio marinofulvus ATCC 14395

Vibrio marinovulgaris ATCC 14394

Vibrio mediterranei ATCC 43341

Vibrio metschnikovii ATCC 7708

Vibrio mytili ATCC 51288

Vibrio natriegens ATCC 14048

Vibrio navarrensis ATCC 51183

Vibrio nereis ATCC 25917

Vibrio nigripulchritudo ATCC 27043

Vibrio ordalii ATCC 33509

Vibrio orientalis ATCC 33933

Vibrio pectenicida ATCC 700783

Vibrio pelagius ATCC 33504

Vibrio penaeicida ATCC 51841

Vibrio ponticus ATCC 14391

Vibrio proteolyticus ATCC 53559

Vibrio psychroerythrus ATCC 27364

Vibrio salmonicida ATCC 43839

Vibrio shiloii ATCC BAA-91

Vibrio splendidus ATCC 33125

Vibrio tyrosinaticus ATCC 19378

Vibrio viscosus ATCC BAA-105

Vibrio wodanis ATCC BAA-104

Beneckea pelagia ATCC 25916

Listonella anguillarum ATCC 19264

Beneckea pelagia and Listonella anguillarum are closely related to the Vibrio bacteria, and some strains thereof are classified into Vibrio bacteria according to the current classification (Thompson F. L. et al., Microbiol. Mol. Biol. Rev., 23, 403-431 (2004) and Macian M. C. et al., Syst. Appl. Microbiol., 23, 373-375 (2000)). Therefore, such bacteria can also be used as the Vibrio bacteria.

Vibrio natriegens is a particular example. Vibrio natriegens is an oceanic facultative anaerobic bacterium belonging to the family Vibrionaceae of γ-Proteobacteria, and it was isolated on 1958 as an uronic acid-oxidizing bacterium (Payne, W. J., J. Bacteriology, 76, 301 (1958)). At first, the bacterium was considered to belong to the genus Pseudomonas, which genus is also of γ-Proteobacteria, but the bacterium was re-classified into the genus Beneckea and then incorporated into the genus Vibrio along with other bacteria belonging to the genus Beneckea. That bacterium is classified as Biosafety level 1 in ATCC, and classified as Risk Group 1 (German classification) in the German National Resource Center for Biological Material (DSMZ), and the pathogenicity is not known for the bacterium.

As Vibrio natriegens, the Vibrio natriegens IFO 15636 strain (ATCC 14048) can be used.

The Vibrio bacteria mentioned above are available from, for example, American Type Culture Collection (Address: P.O. Box 1549, Manassas, Va. 20108, United States of America). That is, registration numbers are given to each of the strains, and the strains can be ordered by using these registration numbers (refer to http://www.atcc.org/). The registration numbers of the strains are listed in the catalogue of the American Type Culture Collection.

The Vibrio bacteria can grow well under high osmotic pressure conditions during the period where the produced substance is produced at a high level at a later stage of amino acid fermentation, or under high osmotic pressure conditions due to a high sugar concentration, in contrast to microorganisms that have been conventionally used for production of an L-amino acid (such as Escherichia coli and coryneform bacteria), which cannot sufficiently grow under such conditions as mentioned above. The “high osmotic pressure” can be, for example, not lower than 925 mOsm, not lower than 1100 mOsm, or even not lower than 1500 mOsm. The maximum osmotic pressure is not particularly limited so long as the amino acid fermentation is possible, and is, for example, 2000 mOsm. In addition, the expression “the bacteria can grow well under high osmotic pressure” can mean that the growth rate is maintained to be, for example, higher than 50% of the maximum growth rate at 1100 mOsm, at which the growth rate of the E. coli wild-type strain, for example, MG1655 strain (ATCC 47076), decreases to 50% or less of the maximum growth rate, or not lower than about 90% of the maximum growth rate.

Methods for imparting an L-amino acid-producing ability to such bacteria as mentioned above and methods for enhancing an L-amino acid-producing ability of such bacteria as mentioned above are described below.

To impart an ability to produce an L-amino acid, methods conventionally employed in the breeding of coryneform bacteria or bacteria of the genus Escherichia (see “Amino Acid Fermentation”, Gakkai Shuppan Center (Ltd.), 1st Edition, published May 30, 1986, pp. 77-100) can be used. Such methods include acquiring an auxotrophic mutant, an L-amino acid analogue-resistant strain, or a metabolic regulation mutant, or constructing a recombinant strain in which an L-amino acid biosynthetic enzyme is overexpressed. In the breeding of L-amino acid-producing bacteria, the above-described property(s) such as auxotrophy, analogue resistance, and metabolic regulation mutation can be imparted alone or in combinations of two or more. The expression of L-amino acid biosynthesis enzyme(s) can be enhanced alone or in combinations of two or more. Furthermore, imparting such properties as an auxotrophy, analogue resistance, or metabolic regulation mutation can be combined with enhancing the biosynthetic enzymes.

An auxotrophic mutant strain, L-amino acid analogue-resistant strain, or metabolic regulation mutant strain with the ability to produce an L-amino acid can be obtained by subjecting a parent or wild-type strain to conventional mutagenesis, such as exposure to X-rays or UV irradiation, or treatment with a mutagen such as N-methyl-N′-nitro-N-nitrosoguanidine, and then selecting those which exhibit autotrophy, analogue resistance, or a metabolic regulation mutation and which also have the ability to produce an L-amino acid.

Moreover, the L-amino acid-producing ability can also be imparted or enhanced by increasing enzymatic activity by gene recombination. An example of the method for increasing enzymatic activity includes modifying the bacterium so that expression of a gene coding for an enzyme involved in biosynthesis of an L-amino acid is enhanced. Gene expression can also be increased by introducing an amplification plasmid prepared by introducing a DNA fragment containing the gene into an appropriate plasmid, for example, a plasmid containing at least a gene responsible for replication and proliferation of the plasmid in the microorganism, increasing the copy number of the gene on the chromosome by conjugation, transfer, or the like, or introducing a mutation into the promoter region of the gene (refer to International Publication WO95/34672).

When an objective gene is introduced into the aforementioned amplification plasmid or chromosome, any promoter can be used to express the gene so long as the chosen promoter functions in bacteria belonging to the Enterobacteriaceae bacteria. The promoter can be the native promoter for the gene, or a modified promoter. The expression of a gene can also be controlled by suitably choosing a promoter that strongly functions in the Enterobacteriaceae bacteria, or by making the −35 and −10 regions of the promoter closer to the consensus sequence. These methods for enhancing expression of enzyme genes are fully described in International Publication WO00/18935, European Patent Laid-open No. 1010755, and so forth.

Specific methods for imparting an L-amino acid-producing ability to bacteria and bacteria imparted with L-amino acid-producing ability are exemplified below.

L-Threonine-Producing Bacteria

Examples of microorganisms having L-threonine-producing ability include bacteria in which one or more activities of L-threonine biosynthetic enzymes are enhanced. Examples of L-threonine biosynthetic enzymes include aspartokinase III (lysC), aspartate semialdehyde dehydrogenase (asd), aspartokinase I (thrA), homoserine kinase (thrB), threonine synthase (thrC), and aspartate aminotransferase (aspartate transaminase) (aspC). The names of the genes coding for the respective enzymes are mentioned in the parentheses after the names of the enzymes (the same shall apply throughout this specification). Among these enzymes, aspartate semialdehyde dehydrogenase, aspartokinase I, homoserine kinase, aspartate aminotransferase, and threonine synthase are particular examples. The genes coding for the L-threonine biosynthetic enzymes can be introduced into an Escherichia bacterium which has a reduced ability to decompose threonine. An example of such an Escherichia bacterium having a reduced ability to decompose threonine is the TDH6 strain, which is deficient in threonine dehydrogenase activity (Japanese Patent Laid-open (Kokai) No. 2001-346578).

The enzymatic activities of the L-threonine biosynthetic enzymes are inhibited by the endproduct, L-threonine. Therefore, for constructing L-threonine-producing strains, the genes for the L-threonine biosynthetic enzymes can be modified so that the enzymes are desensitized to feedback inhibition by L-threonine. The aforementioned thrA, thrB, and thrC genes constitute the threonine operon, which forms an attenuator structure. The expression of the threonine operon is inhibited by isoleucine and threonine in the culture medium and also suppressed by attenuation. Therefore, the threonine operon can be modified by removing the leader sequence in the attenuation region or the attenuator (refer to Lynn, S. P., Burton, W. S., Donohue, T. J., Gould, R. M., Gumport, R. L, and Gardner, J. F., J. Mol. Biol. 194:59-69 (1987); WO02/26993; WO2005/049808).

The native promoter of the threonine operon is present upstream of the threonine operon, and can be replaced with a non-native promoter (refer to WO98/04715), or a threonine operon which has been modified so that expression of the threonine biosynthesis genes is controlled by the repressor and promoter of λ-phage can be constructed (European Patent No. 0593792). Furthermore, in order to modify a bacterium so that it is desensitized to feedback inhibition by L-threonine, a strain resistant to α-amino-β-hydroxyisovaleric acid (AHV) can be selected.

The copy number of the threonine operon that is modified to be desensitized to feedback inhibition by L-threonine can be increased in a host, or the expression of the threonine operon can be increased by ligating it to a potent promoter. The copy number can also be increased by, besides amplification using a plasmid, transferring the threonine operon to a genome by using a transposon, Mu-phage, or the like.

Other than increasing expression of the L-threonine biosynthetic genes, expression of the genes involved in the glycolytic pathway, TCA cycle, or respiratory chain, the genes that regulate the expression of these genes, or the genes involved in sugar uptake can also be increased. Examples of such genes effective on L-threonine production include the genes encoding transhydrogenase (pntAB, European Patent No. 733712), phosphoenolpyruvate carboxylase (ppc, WO95/06114), phosphoenolpyruvate synthase (pps, European Patent No. 877090), and a gene encoding pyruvate carboxylase (pyc) from a coryneform bacterium or a Bacillus bacterium (WO99/18228, European Patent Laid-open No. 1092776).

Expression of a gene that imparts resistance to L-threonine or L-homoserine can be enhanced, or resistance to L-threonine or L-homoserine can be imparted to the host. Examples of such resistance genes include the rhtA gene (Res. Microbiol., 154:123-135 (2003)), rhtB gene (European Patent Laid-open No. 0994190), rhtC gene (European Patent Laid-open No. 1013765), and yfiK and yeaS genes (European Patent Laid-open No. 1016710). The methods for imparting L-threonine resistance to a host are described in European Patent Laid-open No. 0994190 and WO90/04636.

Examples of L-threonine-producing bacteria and parent strains for deriving them include, but are not limited to, strains belonging to the genus Escherichia, such as E. coli TDH-6/pVIC40 (VKPM B-3996) (U.S. Pat. No. 5,175,107, U.S. Pat. No. 5,705,371), E. coli 472T23/pYN7 (ATCC 98081) (U.S. Pat. No. 5,631,157), E. coli NRRL-21593 (U.S. Pat. No. 5,939,307), E. coli FERM BP-3756 (U.S. Pat. No. 5,474,918), E. coli FERM BP-3519 and FERM BP-3520 (U.S. Pat. No. 5,376,538), E. coli MG442 (Gusyatiner et al., Genetika (in Russian), 14, 947-956 (1978)), E. coli VL643 and VL2055 (European Patent Laid-open No. 1149911), and so forth.

The TDH-6 strain is deficient in the thrC gene, and sucrose-assimilative, and the ilvA gene thereof has a leaky mutation. This strain also has a mutation in the rhtA gene, which imparts resistance to high concentration of threonine or homoserine. The B-3996 strain contains the plasmid pVIC40, which was obtained by inserting the thrA*BC operon, including a mutant thrA gene, into the RSF1010-derived vector. This mutant thrA gene encodes aspartokinase homoserine dehydrogenase I which is substantially desensitized to feedback inhibition by threonine. The B-3996 strain was deposited on Nov. 19, 1987 in the All-Union Scientific Center of Antibiotics (Nagatinskaya Street 3-A, 117105 Moscow, Russia) under the accession number RIA 1867. The strain was also deposited at the Russian National Collection of Industrial Microorganisms (VKPM) (1 Dorozhny proezd., 1 Moscow 117545, Russia) on Apr. 7, 1987 under the accession number VKPM B-3996.

E. coli VKPM B-5318 (European Patent No. 0593792) can also be used as an L-threonine-producing bacterium or a parent strain for deriving it. The B-5318 strain is prototrophic with regard to isoleucine, and a temperature-sensitive lambda-phage C1 repressor and PR promoter replace the regulatory region of the threonine operon in the plasmid pVIC40. The VKPM B-5318 strain was deposited as an international deposit at the Russian National Collection of Industrial Microorganisms (VKPM) (1 Dorozhny proezd., 1 Moscow 117545, Russia) on May 3, 1990 under the accession number of VKPM B-5318.

The thrA gene coding for aspartokinase homoserine dehydrogenase I of Escherichia coli has been elucidated (nucleotide numbers 337 to 2799, GenBank accession NC_{—}000913.2, gi: 49175990). The thrA gene is located between the thrL and thrB genes on the chromosome of E. coli K-12. The thrB gene coding for homoserine kinase of Escherichia coli has been elucidated (nucleotide numbers 2801 to 3733, GenBank accession NC_{—}000913.2, gi: 49175990). The thrB gene is located between the thrA and thrC genes on the chromosome of E. coli K-12. The thrC gene coding for threonine synthase of Escherichia coli has been elucidated (nucleotide numbers 3734 to 5020, GenBank accession NC_{—}000913.2, gi: 49175990). The thrC gene is located between the thrB gene and the yaaX open reading frame on the chromosome of E. coli K-12. All these three genes function as a single threonine operon. To increase expression of the threonine operon, the attenuator region, which affects the transcription, can be removed from the operon (WO2005/049808, WO2003/097839).

A mutant thrA gene coding for aspartokinase homoserine dehydrogenase I resistant to feedback inhibition by threonine, as well as the thrB and thrC genes can be obtained as a single operon from the well-known pVIC40 plasmid, which is present in the threonine-producing E. coli strain VKPM B-3996. The plasmid pVIC40 is described in detail in U.S. Pat. No. 5,705,371.

The rhtA gene is present at 18 min on the E. coli chromosome close to the glnHPQ operon, which encodes components of the glutamine transport system. The rhtA gene is identical to ORF1 (ybiF gene, nucleotide numbers 764 to 1651, GenBank accession number AAA218541, gi: 440181) and is located between the pexB and ompX genes. The unit expressing the protein encoded by the ORP1 has been designated the rhtA gene (rht: resistance to homoserine and threonine). Also, it was revealed that the rhtA23 mutation is an A-for-G substitution at position −1 with respect to the ATG start codon (ABSTRACTS of the 17th International Congress of Biochemistry and Molecular Biology in conjugation with Annual Meeting of the American Society for Biochemistry and Molecular Biology, San Francisco, Calif. Aug. 24-29, 1997, abstract No. 457, European Patent Laid-open No. 1013765).

The asd gene coding for aspartate semialdehyde dehydrogenase of E. coli has already been elucidated (nucleotide numbers 3572511 to 3571408, GenBank accession NC_{—}000913.1, gi: 16131307), and can be obtained by PCR (refer to White, T. J. et al., Trends Genet, 5, 185 (1989)) utilizing primers prepared on the basis of the nucleotide sequence of the gene. The asd genes of other microorganisms can also be obtained in a similar manner.

Also, the aspC gene coding for aspartate aminotransferase of E. coli has already been elucidated (nucleotide numbers 983742 to 984932, GenBank accession NC_{—}000913.1, gi: 16128895), and can be obtained by PCR. The aspC genes of other microorganisms can also be obtained in a similar manner.

L-Lysine-Producing Bacteria

Examples of L-lysine-producing bacteria belonging to the genus Escherichia include mutants having resistance to an L-lysine analogue. L-Lysine analogues inhibit the growth of Escherichia bacteria, but this inhibition is fully or partially eliminated when L-lysine is present in the medium. Examples of these L-lysine analogues include, but are not limited to, oxalysine, lysine hydroxamate, S-(2-aminoethyl)-L-cysteine (AEC), γ-methyllysine, α-chlorocaprolactam, and so forth. Mutants having resistance to these lysine analogues can be obtained by subjecting Escherichia bacteria to conventional artificial mutagenesis treatments. Specific examples of bacterial strains useful for producing L-lysine include Escherichia coli AJ11442 (FERM BP-1543, NRRL B-12185; see U.S. Pat. No. 4,346,170) and Escherichia coli VL611. In these microorganisms, aspartokinase is desensitized to the feedback inhibition by L-lysine.

Examples of L-lysine-producing bacteria and parent strains for deriving them also include strains in which expression of one or more genes encoding an L-lysine biosynthetic enzyme are enhanced. Examples of such enzymes include, but are not limited to, dihydrodipicolinate synthase (dapA), aspartokinase (lysC), dihydrodipicolinate reductase (dapB), diaminopimelate decarboxylase (lysA), diaminopimelate dehydrogenase (ddh) (U.S. Pat. No. 6,040,160), phosphoenolpyruvate carboxylase (ppc), aspartate semialdehyde dehydrogenase (asd), succinyl diaminopimelate deacylase (dapE), and aspartase (aspA) (European Patent Laid-open No. 1253195). Among these enzymes, dihydrodipicolinate reductase, diaminopimelate decarboxylase, diaminopimelate dehydrogenase, phosphoenolpyruvate carboxylase, aspartate aminotransferase, diaminopimelate epimerase, and aspartate semialdehyde dehydrogenase are particular examples. In addition, the parent strains can express increased levels of the gene involved in energy efficiency (cyo) (European Patent Laid-open No. 1170376), the genes encoding nicotinamide nucleotide transhydrogenase (pntAB) (U.S. Pat. No. 5,830,716), the ybjE gene, which is a gene for L-lysine secretion (WO2005/073390), or combinations of these.

Examples of L-lysine-producing bacteria and parent strains for deriving them also include strains in which the activity of an enzyme that catalyzes a reaction for generating a compound other than L-lysine, which reaction branches away from the biosynthetic pathway of L-lysine, is reduced or eliminated. Examples of the enzyme that catalyzes a reaction for generating a compound other than L-lysine, which reaction branches away from the biosynthetic pathway of L-lysine, include homoserine dehydrogenase, lysine decarboxylase (U.S. Pat. No. 5,827,698), and the malic enzyme (WO2005/010175).

Examples of L-lysine-producing bacteria include Escherichia coli WC196ΔcadAΔldc/pCABD2 (WO2006/078039). This strain was constructed from the WC1-96 strain by disrupting cadA and ldcC genes, which encode lysine decarboxylase, and introducing the plasmid pCABD2 containing lysine biosynthetic genes (U.S. Pat. No. 6,040,160) into the resulting strain. The WC196 strain was bred from the W3110 strain, which was derived from Escherichia coli K-12, by replacing the wild type lysC gene on the chromosome of the W3110 strain with a mutant lysC gene encoding a mutant aspartokinase III in which threonine at position 352 was replaced with isoleucine, resulting in desensitization of the feedback inhibition thereof by L-lysine (U.S. Pat. No. 5,661,012), and conferring AEC resistance to the resulting strain (U.S. Pat. No. 5,827,698). The WC196 strain was designated Escherichia coli AJ13069, deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Dec. 6, 1994, and assigned an accession number of FERM P-14690. Then, it was converted to an international deposit under the provisions of the Budapest Treaty on Sep. 29, 1995, and assigned an accession number of FERM BP-5252 (U.S. Pat. No. 5,827,698). The WC196ΔcadAΔldc strain itself is also an example of a L-lysine-producing bacterium. The WC196ΔcadAΔldc was designated AJ110692, and deposited at the independent administrative agency, National Institute of Advanced Industrial Science and Technology, International Patent Organism Depositary (Tsukuba Central 6, 1-1, Higashi 1-Chome, Tsukuba-shi, Ibaraki-ken, 305-8566, Japan) on Oct. 7, 2008 as an international deposit and assigned an accession number of FERM BP-11027.

The plasmid pCABD2 contains a mutant dapA gene derived from Escherichia coli and coding for a dihydrodipicolinate synthase (DDPS) having a mutation for desensitization to the feedback inhibition by L-lysine, a mutant lysC gene derived from Escherichia coli and coding for aspartokinase III having a mutation for desensitization to the feedback inhibition by L-lysine, the dapB gene derived from Escherichia coli and coding for dihydrodipicolinate reductase, and the ddh gene derived from Brevibacterium lactofermentum and coding for diaminopimelate dehydrogenase (WO95/16042 and WO01/53459).

As an L-methionine-producing bacterium, an Escherichia bacterium deficient in the repressor of the L-methionine biosynthesis system (metJ) and having enhanced intracellular homoserine transsuccinylase activity (metA), or having attenuated S-adenosylmethionine synthase activity (metK) can be used (Japanese Patent No. 04110641).

L-Tryptophan, L-phenylalanine, and L-tyrosine are all aromatic amino acids and share a common biosynthesis pathway. Examples of the genes encoding the biosynthetic enzymes for aromatic amino acids include genes of deoxyarabino-heptulosonate phosphate synthase (aroG), chorismate mutase/prephenate dehydratase (pheA), 3-dehydroquinate synthase (aroB), shikimate dehydrogenase (aroE), shikimate kinase (aroL), 5-enolpyruvylshikimate-3-phosphate synthase (aroA), and chorismate synthase (aroC) (European Patent Laid-open No. 763127). It is known that these genes are regulated by the tyrosine repressor (tyrR), and so activity of an aromatic amino acid biosynthetic enzyme can also be increased by deleting the tyrR gene (see European Patent No. 763127).

Furthermore, because 3-deoxy-D-arabinoheptulosonate-7-phosphate synthetase (aroF, aroG) is inhibited by feedback inhibition by aromatic amino acids, the enzyme can be modified so as to be desensitized to the feedback inhibition. An aromatic amino acid-producing bacterium can be obtained, for example, by introducing a mutant aroF gene coding for a protein in which the L-aspartic acid at position 147 or the L-serine at position 181, as counted from the N-terminus, is replaced by another amino acid, or by introducing a mutant aroG gene coding for a protein in which single amino acid residue of the L-aspartic acid at position 146, the L-methionine at position 147, the L-proline at position 150, or the L-alanine at position 202, or two amino acid residues of the L-methionine at position 157 and the L-alanine at position 219, as counted from the N-terminus, are replaced by other amino acid(s) (European Patent Laid-open No. 0488424). Furthermore, because the chorismate mutase/prephenate dehydratase is also inhibited by feedback inhibition by aromatic amino acids, it can be modified so as to be desensitized to the feedback inhibition.

Because the aromatic amino acids share a common biosynthesis pathway, a strain in which a biosynthesis system specific to an aromatic amino acid other than the objective L-amino acid is attenuated can be used. For example, a strain that efficiently produces an objective L-amino acid can be obtained by attenuating biosynthesis systems specific to L-phenylalanine or L-tyrosine when the objective amino acid is L-tryptophan, or by attenuating biosynthesis systems specific to L-tryptophan or L-tyrosine when the objective amino acid is L-phenylalanine. Attenuation of a biosynthesis system can be attained by introducing a mutation into a gene coding for an enzyme of the biosynthesis system, or obtaining a strain requiring an L-amino acid synthesized by the biosynthesis system to be attenuated using a synthetic medium containing the L-amino acid (U.S. Pat. No. 4,371,614).

L-Tryptophan-Producing Bacteria

Examples of L-tryptophan-producing bacteria and parent strains for deriving them include, but are not limited to, Escherichia bacteria strains, such as E. coli JP4735/pMU3028 (DSM10122) and E. coli JP6015/pMU91 (DSM10123), which lack tryptophanyl-tRNA synthetase encoded by a mutant trpS gene (U.S. Pat. No. 5,756,345), E. coli AGX17 (pGX44) (NRRL B-12263) and E. coli AGX6(pGX50)aroP (NRRL B-12264), which lack tryptophanase (U.S. Pat. No. 4,371,614), and E. coli AGX17/pGX50, pACKG4-pps, in which phosphoenolpyruvate-producing ability is increased (WO97/08333, U.S. Pat. No. 6,319,696). L-Tryptophan-producing bacteria belonging to the genus Escherichia with increased activity of the protein encoded by the yedA gene or the yddG gene can also be used (U.S. Patent Published Application Nos. 2003/0148473 and 2003/0157667).

Examples of L-tryptophan-producing bacteria and parent strains for deriving them also include strains in which one or two or more activities of the enzymes selected form the following are increased: anthranilate synthase (trpE), phosphoglycerate dehydrogenase (serA), 3-deoxy-D-arabinoheptulosonate-7-phosphate synthase (aroG), 3-dehydroquinate synthase (aroB), shikimate dehydrogenase (aroE), shikimate kinase (aroL), 5-enolpyruvylshikimate-3-phosphate synthase (aroA), chorismate synthase (aroC), prephenate dehydratase, chorismate mutase, and tryptophan synthase (trpAB). The anthranilate synthase and phosphoglycerate dehydrogenase are both inhibited by feedback inhibition by L-tryptophan and L-serine, and therefore a mutation desensitizing the enzymes for the feedback inhibition can be introduced into these enzymes. Specific examples of strains having such a mutation include E. coli SV164(trpE8), which harbors desensitized anthranilate synthase, and SV164(pGH5) obtained by introducing the plasmid pGH5 containing a mutant serA gene encoding feedback inhibition-desensitized phosphoglycerate dehydrogenase into the E. coli SV164.

The aforementioned E. coli SV164(trpE8) is a strain obtained by introducing a mutant trpE gene coding for anthranilate synthase desensitized to the feedback inhibition by L-tryptophan into a trpE-deficient strain, Escherichia coli KB862 (DSM7196) (WO94/08031, Japanese Patent Laid-open (Kohyo) No. 7-507693). The E. coli SV164(pGH5) strain can be obtained by introducing a plasmid pGH5 (WO94/08031) containing a mutant serA5 gene coding for phosphoglycerate dehydrogenase desensitized to feedback inhibition by serine into the SV164 strain. The E. coli SV164(pGH5) strain produces not only L-tryptophan but also L-serine (U.S. Pat. No. 7,045,320).

The aforementioned E. coli KB862 strain was designated AJ13828 and deposited on Dec. 21, 2000 at National Institute of Bioscience and Human Technology of Agency of Industrial Science and Technology (currently independent administrative agency, National Institute of Advanced Industrial Science and Technology, International Patent Organism Depositary, Tsukuba Central 6, 1-1, Higashi 1-Chome, Tsukuba-shi, Ibaraki-ken, 305-8566, Japan) as an international deposit under the provisions of the Budapest Treaty, and assigned an accession number of FERM BP-7405.

Examples of L-tryptophan-producing bacteria and parent strains for deriving them also include a strain which has enhanced activity of 3-phosphoserine phosphatase (serB) (U.S. Pat. No. 4,371,614), a strain which has enhanced activity of phosphoenolpyruvate carboxykinase (pckA) (WO2004/090125), and a strain which constitutively express maleate synthase-isocitrate lyase-isocitrate dehydrogenasekinase/phosphatase operon (ace operon), or of which expression of this operon is enhanced (WO2005/103275).

Examples of L-tryptophan-producing bacteria and parent strains for deriving them also include strains which have been transformed with the tryptophan operon containing a gene encoding inhibition-desensitized anthranilate synthase (Japanese Patent Laid-open Nos. 57-71397, 62-244382, U.S. Pat. No. 4,371,614). Moreover, L-tryptophan-producing ability can be imparted by increasing expression of a gene coding for tryptophan synthase in the tryptophan operon (trpBA). Tryptophan synthase consists of α and β subunits which are encoded by the trpA and trpB genes, respectively. In addition, L-tryptophan-producing ability can be improved by increasing expression of the isocitrate lyase-malate synthase operon (WO2005/103275).

L-Phenylalanine-Producing Bacteria

Examples of L-phenylalanine-producing bacteria and parent strains for deriving them include, but are not limited to, strains belonging to the genus Escherichia, such as E. coli AJ12479 (FERM BP-4796) (European Patent Laid-open No. 1484410), E. coli AJ12739 deficient in chorismate mutase-prephenate dehydratase and tyrocine repressor (tyrA::Tn10, tyrR) (VKPM B-8197), E. coli HW1089 (ATCC 55371) harboring a mutant pheA34 gene coding for chorismate mutase-prephenate dehydratase desensitized to feedback inhibition (U.S. Pat. No. 5,354,672), E. coli MWEC101-b (KR8903681), E. coli NRRL B-12141, NRRL B-12145, NRRL B-12146, and NRRL B-12147 (U.S. Pat. No. 4,407,952). As a parent strain, E. coli K-12 [W3110 (tyrA)/pPHAB] (FERM BP-3566) harboring a gene coding for chorismate mutase-prephenate dehydratase desensitized to the feedback inhibition, E. coli K-12 [W3110 (tyrA)/pPHAD] (FERM BP-12659), E. coli K-12 [W3110 (tyrA)/pPHATerm] (FERM BP-12662) and E. coli K-12 [W3110 (tyrA)/pBR-aroG4, pACMAB] named as AJ12604 (FERM BP-3579) can also be used (European Patent No. 488424). Furthermore, L-phenylalanine-producing bacteria belonging to the genus Escherichia with an increased activity of the protein encoded by the yedA gene or the yddG gene can also be used (U.S. Patent Published Application Nos. 2003/0148473 and 2003/0157667).

As for phenylalanine-producing bacteria, strains efficiently producing L-phenylalanine can also be obtained by such modification that bacteria incorporate by-products into cells, for example, by increasing expression amount of the L-tryptophan uptake gene, tnaB or mtr, or the L-tyrosine uptake gene, tyrP (European Patent No. 1484410).

L-Tyrosine-Producing Bacteria

Examples of tyrosine-producing bacteria include Escherichia bacteria having a desensitized type prephenate dehydratase gene (tyrA), of which the product is not inhibited by tyrosine (European Patent Laid-open No. 1616940).

When the bacterium is bred by gene recombination, the genes to be used are not limited to genes having the genetic information described above or genes having known sequences, but also include variants of the genes, namely, genes having conservative mutations, such as homologues of the genes or artificially modified genes, can also be used so long as the functions of the encoded proteins are not degraded. That is, they can be genes encoding a protein having a known amino acid sequence, but including substitution, deletion, insertion, addition, or the like of one or several amino acid residues at one or several positions.

Although the number of the “one or several” referred to herein can differ depending on the position of amino acid residues in the three-dimensional structure of the protein or the types of amino acid residues, specifically, it can be 1 to 10, 1 to 5, 1 to 3, or even 1 or 2. The conservative mutation is typically a conservative substitution. The conservative substitution is a mutation wherein substitution takes place mutually among Phe, Trp, and Tyr, if the substitution site is an aromatic amino acid; among Leu, Ile and Val, if it is a hydrophobic amino acid; between Gln and Asn, if it is a polar amino acid; among Lys, Arg and His, if it is a basic amino acid; between Asp and Glu, if it is an acidic amino acid; and between Ser and Thr, if it is an amino acid having a hydroxyl group. Substitutions considered conservative substitutions include, specifically, substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Gly, Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr, and substitution of Met, Ile or Leu for Val. The aforementioned amino acid substitution, deletion, insertion, addition, inversion, or the like can be a result of a naturally-occurring mutation or a variation due to an individual difference or difference of species of a microorganism from which the genes are derived (mutant or variant). Such genes can be obtained by, for example, modifying a known nucleotide sequence of a gene by site-specific mutagenesis so that the amino acid residues at the specific sites of the encoded protein include substitution, deletion, insertion, or addition of amino acid residues.

Furthermore, such genes having conservative mutation(s) as described above can encode a protein having a homology of 80% or more, 90% or more, 95% or more, or even 97% or more, to the entire encoded amino acid sequence and having a function equivalent to that of the wild-type protein. In this specification, “homology” can mean “identity”.

Moreover, codons in the gene sequences can be replaced with other codons which are easily used in the host into which the genes are introduced.

The genes having conservative mutation(s) can be obtained by methods usually used in mutagenesis treatments such as treatments with mutagenesis agents.

Furthermore, the genes can be a DNA which can hybridize with a complementary sequence of a known gene sequence or a probe which can be prepared from such a complementary sequence under stringent conditions and encodes a protein having a function equivalent to that of the known gene product. The “stringent conditions” referred to here can be conditions under which a so-called specific hybrid is formed, and a non-specific hybrid is not formed. Examples of the stringent conditions include those under which highly homologous DNAs hybridize to each other, for example, DNAs not less than 80% homologous, not less than 90% homologous, not less than 95% homologous, not less than 97% homologous, hybridize to each other, and DNAs less homologous than the above do not hybridize to each other, or conditions of washing of typical Southern hybridization, i.e., conditions of washing once, 2 or 3 times, at a salt concentration and temperature corresponding to 1×SSC, 0.1% SDS at 60° C., 0.1×SSC, 0.1% SDS at 60° C., or even 0.1×SSC, 0.1% SDS at 68° C.

As the probe, a part of a complementary sequence of a gene can also be used. Such a probe can be prepared by PCR using oligonucleotides prepared on the basis of a known gene sequence as primers and a DNA fragment containing the nucleotide sequences as a template. For example, when a DNA fragment having a length of about 300 bp is used as the probe, the washing conditions of hybridization can be 50° C., 2×SSC and 0.1% SDS.

The aforementioned descriptions concerning variants of genes are similarly applied to the ydgT gene described below and the other genes mentioned in this specification.

The bacterium can have glycerol-assimilating ability, and it can inherently have glycerol-assimilating ability, or a recombinant strain imparted with glycerol-assimilating ability.

The L-amino acid-producing bacterium can be modified so that glycerol-assimilating ability thereof is enhanced. The glycerol-assimilating ability can be imparted or enhanced by modifying a gene involved in the glycerol metabolism.

In order to enhance glycerol-assimilating ability, expression of the glpR gene (European Patent No. 1715056) can be attenuated, or expression of a glycerol metabolism gene such as glpA, glpB, glpC, glpD, glpE, glpF, glpG, glpK, glpQ, glpT, glpX, tpiA, gldA, dhaK, dhaL, dhaM, dhaR, fsa and talC genes (European Patent Laid-open No. 1715055) can be enhanced. In particular, in order to enhance glycerol-assimilating property, expression of the glycerol dehydrogenase gene (gldA), dihydroxyacetone kinase gene (dhaKLM, dak), and fructose-6-phosphate aldolase gene (fsaB) can be enhanced (WO2008/102861).

Furthermore, as for glycerol kinase (glpK), a desensitized type glpK gene of which the product is desensitized to the feedback inhibition by fructose-1,6-bisphosphate (WO2008/081959, WO2008/107277) can be used.

<1-2> Reduction of Activity of Cnu Protein

Hereafter, the modification for reducing the activity of the Cnu protein is described.

The Cnu protein is encoded by the ydgT gene (synonyms: Cnu, ECK1621, b1625). Specific examples include, as the ydgT gene of Escherichia coli, a gene having the nucleotide sequence shown in SEQ ID NO: 13, which is located at the nucleotide numbers 1702973 to 1703188 of the genome sequence of Escherichia coli (GenBank Accession No. U00096). The amino acid sequence encoded by this gene is shown in SEQ ID NO: 14.

The ydgT gene product is also known as B1625, YdgT, or H-NS- and StpA-binding protein, and it has been reported that the Cnu protein binds to the replication origin oriC (Kim, M. S. et al., J. Bacteriol., 187:6998-7008 (2005)).

The “activity of the Cnu protein” can be such a property that when the activity is reduced in a bacterium, for example, when amount of the Cnu protein in the cell is reduced, an L-amino acid-producing ability, for example, L-lysine-producing ability, can be improved compared with that of a non-modified strain, or proliferation rate or glycerol-assimilating ability of the bacterium can be improved.

Examples of the Cnu protein include, for example as for Escherichia coli, a protein having the amino acid sequence of SEQ ID NO: 14. However, the Cnu protein can also be a protein having the amino acid sequence of SEQ ID NO: 14, but including a conservative mutation(s) so long as the protein has the activity of the Cnu protein. That is, the Cnu protein can be a protein having the amino acid sequence of SEQ ID NO: 14, but including substitution, deletion, insertion, addition, or the like of one or several amino acid residues.

Although the number of the “one or several” referred to herein can differ depending on the position of amino acid residues in the three-dimensional structure or the types of amino acid residues of the protein, specifically, it can be, for example, 1 to 20, 1 to 10, or even 1 to 5. The conservative mutation is typically a conservative substitution. The conservative substitution is a mutation wherein substitution takes place mutually among Phe, Trp, and Tyr, if the substitution site is an aromatic amino acid; among Leu, Ile and Val, if it is a hydrophobic amino acid; between Gln and Asn, if it is a polar amino acid; among Lys, Arg and His, if it is a basic amino acid; between Asp and Glu, if it is an acidic amino acid; and between Ser and Thr, if it is an amino acid having a hydroxyl group. Substitutions considered conservative substitutions include, specifically, substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr, and substitution of Met, Ile or Leu for Val. The aforementioned amino acid substitution, deletion, insertion, addition, inversion, or the like can be a result of a naturally-occurring mutation or a variation due to an individual difference or difference of species of a microorganism from which the genes are derived (mutant or variant). Such genes can be obtained by, for example, modifying a known nucleotide sequence of a gene by site-specific mutagenesis so that the amino acid residues at the specific sites of the encoded protein include substitution, deletion, insertion, or addition of amino acid residues.

The gene coding for the Cnu protein can be a DNA that is able to hybridize with a nucleotide sequence complementary to the nucleotide sequence of SEQ ID NO: 13 or a probe that can be prepared from the nucleotide sequence under stringent conditions, so long as it codes for the Cnu protein. The “stringent conditions” can refer to conditions under which a so-called specific hybrid is formed, and a non-specific hybrid is not formed. Examples of the stringent conditions include those under which highly homologous DNAs hybridize to each other, for example, DNAs not less than 90% homologous, not less than 95% homologous, not less than 97% homologous, or not less than 99% homologous, hybridize to each other, and DNAs less homologous than the above do not hybridize to each other, or conditions of washing of typical Southern hybridization, i.e., conditions of washing once, or 2 or 3 times, at a salt concentration and temperature corresponding to 1×SSC, 0.1% SDS at 60° C., 0.1×SSC, 0.1% SDS at 60° C., or 0.1×SSC, 0.1% SDS at 68° C.

As the probe, a part of a sequence that is complementary to the gene can also be used. Such a probe can be prepared by PCR using oligonucleotides prepared on the basis of a known gene sequence as primers, and a DNA fragment containing the nucleotide sequence as a template. Although length of the probe is appropriately chosen according to the conditions of the hybridization, it can be 100 bp to 1 kbp. When a DNA fragment having a length of about 300 bp is used as the probe, the washing conditions of the hybridization can be, for example, 50° C., 2×SSC and 0.1% SDS.

The phrase “modified so that the activity of the Cnu protein is reduced” can mean that the activity of the Cnu protein per cell of the bacterium has become lower than that of a non-modified strain, such as a wild-type strain of the bacterium belonging to the family Enterobacteriaceae. This means, for example, that the number of molecules of the Cnu protein per cell is decreased, or that the activity of the Cnu protein per molecule is decreased. The term “reduction” of the activity includes the complete disappearance of the activity.

The phrase “the activity of the Cnu protein is reduced” can mean that activity of the Cnu protein is reduced to, for example, 50% or less, 30% or less, or 10% or less, of the activity of a non-modified strain, such as a wild-type strain. Examples of the wild-type strain of Escherichia bacterium as a control of the comparison include, for example, the Escherichia coli MG1655 strain, and so forth.

The modification for reducing the activity of the Cnu protein can be achieved by, specifically, deleting a part or all of the ydgT gene on a chromosome, or modifying an expression control sequence such as a promoter or the Shine-Dalgarno (SD) sequence of the gene. The reduction of expression includes reduction of transcription and reduction of translation. Furthermore, expression of the gene can also be reduced by modifying a non-translation region other than the expression control sequence. The entire gene as well as the sequences on both sides of the gene on the chromosome can also be deleted. Furthermore, the modification can also be attained by introducing an amino acid substitution (missense mutation), a stop codon (nonsense mutation), or a frame shift mutation which adds or deletes one or two nucleotides, into the coding region of the gene on the chromosome (Journal of Biological Chemistry, 272:8611-8617 (1997); Proceedings of the National Academy of Sciences, USA, 95:5511-5515 (1998); Journal of Biological Chemistry, 266, 20833-20839 (1991)).

Modifying a gene so that the function of the gene or the expression product thereof is decreased or deleted as described above is also called gene disruption. Disruption of the gene of the Cnu protein can be performed by deleting the entire sequence or 30% or more, 50% or more, or 70% or more, of the promoter and/or the coding region of the gene.

Modification of the gene can be attained by gene recombination. Specific examples of the methods based on gene recombination include deleting a part or all of an expression control sequence such as a promoter region, a coding region, or a non-coding region of a target gene on the chromosome, inserting another sequence into these regions, and introducing a frame shift mutation, a nonsense mutation, or a missense mutation into these regions, by utilizing homologous recombination.

A modification of an expression control sequence can be performed for one or more nucleotides, two or more nucleotides, or three or more nucleotides. When a coding region is deleted, the region to be deleted can be any of an N-terminus region, an internal region, or a C-terminus region, or even can be the entire coding region, so long as the function of the protein produced from the gene is decreased or deleted. Deletion of a longer region can usually more surely inactivate a target gene. Furthermore, reading frames upstream and downstream of the region to be deleted can be different.

Also when another sequence is inserted into the coding region, such a sequence can be inserted into any region of the target gene, and insertion of a longer sequence can more surely inactivate the target gene. The reading frames upstream and downstream of the insertion site can be different. The other sequence to be inserted is not particularly limited so long as the chosen sequence decreases or deletes the function of a protein encoded by the target gene, and examples include a transposon carrying an antibiotic resistance gene or a gene useful for L-amino acid production, and so forth.

A target gene on the chromosome can be modified as described above by, for example, preparing a deletion-type gene in which a partial sequence of the gene is deleted so that the deletion-type gene does not produce a protein that normally functions, and transforming a bacterium with a DNA containing the deletion-type gene to cause homologous recombination between the deletion-type gene and the target gene on the chromosome, and thereby substitute the deletion-type gene for the target gene on the chromosome. The protein encoded by the deletion-type gene has a conformation different from that of the wild-type protein, if it is even produced, and thus the function is reduced or deleted. Such gene disruption based on gene substitution utilizing homologous recombination has been already established, and there are a method called “Red-driven integration” (Datsenko, K. A, and Wanner, B. L., Proc. Natl. Acad. Sci. USA, 97:6640-6645 (2000)), a method using a linear DNA such as a method utilizing the Red driven integration in combination with an excision system derived from λ phage (Cho, E. H., Gumport, R. I., Gardner, J. F., J. Bacteriol., 184:5200-5203 (2002)) (refer to WO2005/010175), a method using a plasmid containing a temperature sensitive replication origin, a method using a plasmid capable of conjugative transfer, a method utilizing a suicide vector not having replication origin in a host (U.S. Pat. No. 6,303,383, Japanese Patent Laid-open (Kokai) No. 05-007491), and so forth.

Decrease of the transcription amount of the target gene can be confirmed by comparing the amount of mRNA transcribed from the target gene with that observed in a wild-type strain or non-modified strain. Examples of the method for evaluating mRNA amount include Northern hybridization, RT-PCR (Molecular Cloning, Cold Spring Harbor Laboratory Press, Cold Spring Harbor, USA, 2001), and so forth. Although the transcription amount can be decreased to any extent so long as it is decreased compared with that observed in a wild-type strain or non-modified strain, it can be decreased to, for example, at least 75% or less, 50% or less, 25% or less, or 10% or less, of that observed in a wild-type strain or non-modified strain, and the gene may not be expressed at all.

Decrease in amount of the protein encoded by the target gene can be confirmed by Western blotting using antibodies that bind to the protein (Molecular Cloning, Cold Spring Harbor Laboratory Press, Cold Spring Harbor, USA, 2001). Although the amount of the protein can be decreased to any extent so long as it is decreased compared with that observed in a wild-type strain or non-modified strain, it can be decreased to, for example, at least 75% or less, 50% or less, 25% or less, or 10% or less, of that observed in a wild-type strain or non-modified strain, and the protein may not be produced at all (the activity has completely disappeared).

A gene coding for the Cnu protein showing low activity can also be obtained by subjecting the ydgT gene to a mutation treatment.

Examples of the method for reducing the activity of the Cnu protein include, besides the aforementioned genetic manipulation techniques, for example, a method of treating an Escherichia bacterium with ultraviolet irradiation or a mutagen used for usual mutagenesis treatment such as N-methyl-N′-nitro-N-nitrosoguanidine (NTG) or nitrous acid, and selecting a strain showing reduced activity of the Cnu protein.

Method for Producing L-Amino Acid

A bacterium belonging to γ-Proteobacteria, having the ability to produce an L-amino acid, and modified so that the activity of the Cnu protein is reduced is cultured in a medium containing glycerol as a carbon source to produce and accumulate the L-amino acid in the medium, and the L-amino acid is collected from the medium. Examples of the L-amino acid include the aforementioned L-amino acids, especially aspartic acid type amino acids and aromatic amino acids, and among these, L-lysine is a particular example.

Glycerol can be used as a carbon source. Glycerol can be used at any concentration so long as a concentration suitable for production of the L-amino acid is chosen. When glycerol is used as a sole carbon source in the medium, it can be present in the medium in an amount of about 0.1 to 50 w/v %, about 0.5 to 40 w/v %, or about 1 to 30% w/v %. Glycerol can also be used in combination with other carbon sources such as glucose, fructose, sucrose, blackstrap molasses, and starch hydrolysate. In this case, although glycerol and other carbon sources can be mixed at an arbitrary ratio, it is desirable that the ratio of glycerol in the carbon source should be 10% by weight or more, 50% by weight or more, or 70% by weight or more. The other carbon sources can be saccharides such as glucose, fructose, sucrose, lactose, galactose, blackstrap molasses, starch hydrolysate, and a sugar solution obtained by hydrolysis of biomass, alcohols such as ethanol, and organic acids such as fumaric acid, citric acid and succinic acid. Among these, glucose is one example.

Although initial concentration of glycerol at the time of starting the culture can be as described above, glycerol can be supplemented according to the consumption of glycerol during the culture.

Glycerol can be pure glycerol or crude glycerol. Crude glycerol refers to industrially produced glycerol containing impurities. Crude glycerol is industrially produced by contacting oils with water under a high temperature and high pressure to hydrolyze them, or by the esterification reaction for biodiesel fuel production. Biodiesel fuel refers to fatty acid methyl esters produced from oils and methanol by a transesterification reaction, and crude glycerol is produced as a by-product of this reaction (refer to Fukuda, H., Kondo, A., and Noda, H., 2001, J. Biosci. Bioeng., 92, 405-416). In the biodiesel fuel production process, the alkaline catalyst method is used for the transesterification in many cases, and acids are added for neutralization. Therefore, crude glycerol of a purity of about 70 to 95% by weight, containing water and impurities, is produced. Crude glycerol produced in the biodiesel fuel production process contains, in addition to water, residual methanol and salts of alkali such as NaOH as a catalyst and an acid used for neutralization thereof such as H₂SO₄ as impurities. Although it depends on manufacturers and production methods, the amount of these salts and methanol can reach several percent. The crude glycerol can contain ions derived from the alkali and the acid used for neutralization thereof, such as sodium ions, potassium ions, chloride ions, and sulfate ions, in an amount of 2 to 7%, 3 to 6%, or 4 to 5.8%, based on the weight of the crude glycerol. Although methanol may not be present as an impurity, it can be present in an amount of 0.01% or less.

The crude glycerol can further contain trace amounts of metals, organic acids, phosphorus, fatty acids, and so forth. Examples of the organic acids include formic acid, acetic acid, and so forth, and although they may not be present as impurities, they can be present in an amount of 0.01% or less. As the trace metals contained in the crude glycerol, trace metals required for growth of the microorganism can be present, and examples include, for example, magnesium, iron, calcium, manganese, copper, zinc, and so forth. Magnesium, iron, and calcium can be present in an amount of 0.00001 to 0.1%, 0.0005 to 0.1%, 0.004 to 0.05%, or even 0.007 to 0.01%, in terms of the total amount based on the weight of the crude glycerol. Manganese, copper, and zinc can be present in an amount of 0.000005 to 0.01%, 0.000007 to 0.005%, or 0.00001 to 0.001%, in terms of the total amount.

It is sufficient that the purity of glycerol in the crude glycerol is 10% or higher, and can be 50% or higher, 70% or higher, or even 80% or higher. As long as the amounts of impurities are within the aforementioned ranges, the purity of the glycerol can be 90% or higher.

When crude glycerol is used, the crude glycerol can be added to the medium according to the purity of the glycerol so that the amount of glycerol is within the concentration range described above. Both glycerol and crude glycerol can be added to the medium.

As the medium, ordinary media containing a nitrogen source, inorganic ions, and other organic components as required, in addition to the aforementioned carbon source, can be used. As the nitrogen source in the medium, ammonia, ammonium salts such as ammonium sulfate, ammonium carbonate, ammonium chloride, ammonium phosphate, ammonium acetate, and urea, nitrate salts, and so forth can be used. Ammonia gas or aqueous ammonia used for adjustment of pH can also be used as the nitrogen source. Peptone, yeast extract, meat extract, malt extract, corn steep liquor, soybean hydrolysate, and so forth can also be used as the nitrogen source. The medium can contain one of these nitrogen sources, or two or more of them. These nitrogen sources can be used in both starting medium and feed medium. Furthermore, the same nitrogen source can be used in both the starting medium and feed medium, or nitrogen source of the feed medium can be different from nitrogen source of the starting medium.

The medium can contain a phosphate source and a sulfur source in addition to the carbon source and the nitrogen source. As the phosphate source, potassium dihydrogen phosphate, dipotassium hydrogen phosphate, phosphate polymers such as pyrophosphoric acid and so forth can be utilized. Although the sulfur source can be any substance containing sulfur atoms, sulfate salts such as sulfates, thiosulfates, and sulfites, and sulfur-containing amino acids such as cysteine, cystine, and glutathione can be used, and ammonium sulfate is a particular example.

Furthermore, the medium can contain a growth-promoting factor (nutrient having a growth promoting effect) in addition to the carbon source, nitrogen source, and sulfur source. As the growth promoting factor, trace metals, amino acids, vitamins, nucleic acids as well as peptone, casamino acid, yeast extract, soybean protein degradation product, and so forth containing the foregoing substances can be used. Examples of the trace metals include iron, manganese, magnesium, calcium, and so forth. Examples of the vitamins include vitamin B₁, vitamin B₂, vitamin B₆, nicotinic acid, nicotinamide, vitamin B₁₂, and so forth. These growth-promoting factors can be present in the starting medium or the feed medium.

Furthermore, when an auxotrophic mutant strain that requires an amino acid or the like for growth thereof is used, it is preferable to supplement a required nutrient to the medium. In particular, because the L-lysine biosynthetic pathway is often enhanced and L-lysine-degrading ability is often attenuated in L-lysine-producing bacteria that can be used for the present invention as described above, one or more substances selected from L-threonine, L-homoserine, L-isoleucine, and L-methionine are preferably added. The starting medium and the feed medium can have the same medium composition or different medium compositions. Furthermore, when the feed medium is fed at multiple steps, the composition of the feed medium fed at each of the steps can be the same or different.

The culture can be performed as aeration culture at a fermentation temperature of 20 to 45° C., or 33 to 42° C. The culture can be performed with controlling the oxygen concentration to be about 5 to 50%, or about 10%. Furthermore, pH can be controlled to be 5 to 9 during the aeration culture. If pH of the medium is lowered during the culture, for example, calcium carbonate can be added, or the medium can be neutralized with an alkaline such as ammonia gas and aqueous ammonia. If culture is performed under such conditions as described above for about 10 to 120 hours, a marked amount of L-amino acid can be accumulated in the culture medium. Although the concentration of L-amino acid accumulated is not limited so long as it is higher than that observed with a wild-type strain, and it enables isolation and collection of the L-amino acid from the medium, it can be 50 g/L or higher, 75 g/L or higher, or 100 g/L or higher.

In order to keep the accumulation of L-amino acid at a certain level or higher, culture of the microorganism can be carried out as separate seed culture and main culture. The seed culture can be carried out with shaking using a flask or the like or as batch culture, and the main culture can be carried out as fed-batch culture or continuous culture. Both the seed culture and main culture can be carried out as batch culture.

When the objective amino acid is a basic amino acid, the production can be performed by a method in which fermentation is performed by controlling pH of the medium during culture to be 6.5 to 9.0 and pH of the medium at the end of the culture to be 7.2 to 9.0 and controlling the pressure in the fermentation tank to be positive during the fermentation, or by supplying carbon dioxide gas or a mixed gas containing carbon dioxide gas to the medium to provide a culture period where the medium contains 2 g/L or more of bicarbonate ions and/or carbonate ions, so that these bicarbonate ions and/or carbonate ions act as counter ions of cations mainly consisting of a basic amino acid, and the objective basic amino acid is then collected (refer to Japanese Patent Laid-open (Kokai) No. 2002-065287).

The L-amino acid can be collected from fermentation broth by a combination of conventionally known methods such as ion-exchange resin method (Nagai, H. et al., Separation Science and Technology, 39(16), 3691-3710), precipitation, membrane separation (Japanese Patent Laid-open (Kokai) Nos. 9-164323 and 9-173792), crystallization (WO2008/078448, WO2008/078646), and other methods. When the L-amino acid accumulates in the cells, the cells can be disrupted with, for example, ultrasonic waves or the like, and the L-amino acid can be collected by the ion exchange resin method or the like from the supernatant obtained by removing the cells from the cell-disrupted suspension by centrifugation.

The L-amino acid collected can contain bacterial cells, medium components, moisture, and by-product metabolites of the bacterium in addition to the objective L-amino acid. Purity of the collected L-amino acid can be 50% or higher, 85% or higher, or 95% or higher (Japanese Patent No. 1214636, U.S. Pat. Nos. 5,431,933, 4,956,471, 4,777,051, 4,946,654, 5,840,358, 6,238,714, U.S. Patent Published Application No. 2005/0025878).

Furthermore, when L-amino acid precipitates in the medium, it can be collected by centrifugation, filtration, or the like. L-Amino acid precipitated in the medium and L-amino acid dissolved in the medium can be isolated together after the L-amino acid dissolved in the medium is crystallized.

Phenylalanine produced by the method can be used for, for example, producing a lower alkyl ester of α-L-aspartyl-L-phenylalanine (also referred to as “aspartame”). Therefore, a method is provided for producing a lower alkyl ester of α-L-aspartyl-L-phenylalanine using L-phenylalanine as a starting material. This method includes the step of synthesizing a lower alkyl ester of α-L-aspartyl-L-phenylalanine from L-phenylalanine produced by the aforementioned method, and aspartic acid or a derivative thereof. Examples of the lower alkyl ester include methyl ester, ethyl ester, propyl ester, and so forth.

The method for synthesizing a lower alkyl ester of α-L-aspartyl-L-phenylalanine from L-phenylalanine and aspartic acid or a derivative thereof is not particularly limited, so long as L-phenylalanine or a derivative thereof is used for the synthesis of the lower alkyl ester of α-L-aspartyl-L-phenylalanine. Specifically, for example, a lower alkyl ester of α-L-aspartyl-L-phenylalanine can be produced by the following method (U.S. Pat. No. 3,786,039). L-Phenylalanine is esterified to obtain a lower alkyl ester of L-phenylalanine. The L-phenylalanine alkyl ester is reacted with an L-aspartic acid derivative of which β-carboxyl group is protected and α-carboxyl group is esterified and thereby activated. Examples of such a derivative include N-acyl-L-aspartic anhydride such as N-formyl-, N-carbobenzoxy-, or N-p-methoxycarbobenzoxy-L-aspartic anhydride. By this condensation reaction, a mixture of N-acyl-α-L-aspartyl-L-phenylalanine and N-acyl-β-L-aspartyl-L-phenylalanine is obtained. If the condensation reaction is performed in the presence of an organic acid of which acid dissociation constant at 37° C. is 10⁻⁴ or less, the ratio of the α-isomer to the β-isomer in the mixture is increased (Japanese Patent Laid-Open (kokai) No. 51-113841). Then, the N-acyl-α-L-aspartyl-L-phenylalanine is separated from the mixture, and hydrogenated to obtain α-L-aspartyl-L-phenylalanine.

### EXAMPLES

Hereafter, the present invention will be still more specifically explained with reference to examples.

### Example 1

### Deletion of ydgT Gene in Escherichia coli W3110 Strain

The ydgT gene on the chromosome of the Escherichia coli W3110 strain was deleted by the method (WO2005/010175) which includes the combination of the method of Datsenko et al. (Datsenko, K. A. et al., Proc. Natl. Acad. Sci. USA, 97:6640-6645 (2000)) and an excision system derived from λ phage (Cho E. H. et al., J. Bacteriol., 184:5200-5203 (2002)).

Specifically, PCR was performed by using the plasmid pMW118-attL-cat-attR (WO2005/010175) as a template DNA and synthetic oligo-DNAs shown in SEQ ID NOS: 1 and 2. pMW118-attL-cat-attR is obtained by inserting the attL and attR genes, which are the attachment sites of λ phage, and the cat gene, which is an antibiotic resistance gene, into pMW118 (Takara Bio) in the order of attL-cat-attR.

After PCR, the template DNA was fully digested with the restriction enzyme DpnI, and the obtained PCR product was purified with Wizard® SV Gel and PCR Clean-Up System (Promega, USA). This PCR product was introduced into the Escherichia coli W3110 strain containing the plasmid pKD46 having temperature sensitive replication ability (WO2005/010175, Datsenko, K. A. et al., Proc. Natl. Acad. Sci. USA., 97:6640-6645 (2000)) by electroporation. The plasmid pKD46 is a temperature sensitive plasmid containing the DNA fragment of 2154 nucleotides in total of λ phage (GenBank/EMBL accession number J02459, 31088th to 33241st nucleotides) containing the genes coding for the Red recombinase of λRed homologous recombination system (γ, β and exo genes) controlled by the arabinose-inducible ParaB promoter. In order to prepare competent cells for electroporation, W3110/pKD46 was cultured at 30° C. for 18 hours in 1 mL of the LB medium containing 100 mg/L of ampicillin. The obtained culture broth (300 μl) was added to the LB medium (50 mL) containing 100 mg/L of ampicillin and 1 mM L-arabinose, and flask culture was performed at 30° C. for 3 hours. The obtained cells were washed 3 times with 10% glycerol to prepare competent cells for electroporation. The PCR product (about 100 ng) was added to 300 μL of the competent cells, and electroporation was performed. The cells subjected to electroporation were cultured at 37° C. for 1 hour in 1 mL of the LB medium, and then inoculated on the LB agar medium (containing 40 mg/L of chloramphenicol). The cells were cultured at 37° C. for 18 hours to obtain a chloramphenicol resistant strain, and thereby obtain a W3110 strain in which the ydgT gene on the chromosome was replaced with the attL-cat-attR gene. This strain was designated W3110ydgT::cat strain.

Then, in order to remove the attL-cat-attR gene from the W3110ydgT::cat strain, the helper plasmid pMW-intxis-ts (WO2005/010175) was used. pMW-intxis-ts is a plasmid carrying a gene coding for λ phage integrase (Int) and a gene coding for excisionase (Xis), and having temperature sensitive replication ability. If pMW-intxis-ts is introduced into a cell, recombination occurs between the plasmid and attL or attR on the chromosome, thus the gene between attL and attR is excised, and only the attL or attR sequence remains on the chromosome.

Competent cells of the W3110ydgT::cat strain were prepared in a conventional manner, transformed with the helper plasmid pMW-intxis-ts, and cultured at 30° C. for 24 hours on the LB agar medium containing 100 mg/L of ampicillin to select an ampicillin-resistant strain. Then, to remove the pMW-intxis-ts plasmid, the ampicillin-resistant transformant was inoculated on the LB agar medium and cultured at 37° C. for 18 hours. Ampicillin resistance and chloramphenicol resistance of the obtained colonies were examined to obtain a ydgT gene-disrupted strain in which attL-cat-attR and pMW-intxis-ts had been eliminated, W3110ΔydgT. Disruption of the ydgT gene on the chromosome was confirmed by PCR using the synthetic DNAs shown in SEQ ID NOS: 7 and 8 as primers. Then, in order to impart an antibiotic resistance marker to the W3110ΔydgT strain, competent cells of W3110ΔydgT were produced in a conventional manner, and pSTV29 was introduced.

### Example 2

### L-Lysine Production with Escherichia coli L-Lysine-Producing Bacterium Deficient in ydgT Gene (1)

L-Lysine production was performed by using Escherichia coli deficient in the ydgT gene. As the L-lysine-producing bacterium, the WC196ΔcadAΔldcC strain (also called “WC196LC strain”, WO96/17930, European Patent No. 0796912) was used.

The ydgT gene on the chromosome of the WC196ΔcadAΔldcC strain was disrupted by P1 transduction using the W3110ydgT::cat strain, specifically, as follows. The W3110ydgT::cat strain was cultured at 37° C. for 18 hours in 1 mL of the LB medium. The obtained culture broth (50 μL) was added to the LB medium (4 mL), and culture was performed at 37° C. for 1 hour. The cells were collected from the culture broth, and suspended in fresh LB medium (1 mL, containing 5 mM calcium chloride). P1 phage was added to the cell suspension, and the suspension was left at 37° C. for 1 hour. To the cell suspension infected with the P1 phage, 2.5 mL of R-Top Agar (containing 10 g/L of tryptone, 1 g/L of yeast extract, 8 g/L of NaCl, 8 g/L of agar, 2 mM CaCl₂, and 0.1% glucose) was added, and the mixture was overlaid on the LB agar medium, and incubated at 37° C. for 14 hours. Then, 2 mL of the LB medium was added on the plate, the P1 phage and the cells were collected together with R-Top Agar, 30 μL of chloroform was added to them, and the supernatant was collected by centrifugation. After the addition of chloroform and collection of supernatant were repeated 3 times, the supernatant was filtered with a filter having a pore size of 0.2 μm to obtain a P1 phage solution for ydgT gene disruption.

Then, the WC196ΔcadAΔldcC strain was cultured at 37° C. for 18 hours in 1 mL of the LB medium. The obtained culture broth (50 μl) was added to 4 mL of the LB medium, and culture was performed at 37° C. for 1 hour. The cells were collected from the obtained culture broth, and suspended in fresh LB medium (1 mL, containing 5 mM calcium chloride), the P1 phage solution for ydgT gene disruption was added to the suspension, and the mixture was left at room temperature for 15 minutes. To the suspension, 5 mL of the LB medium containing 1% citric acid was added, the mixture was left at 30° C. for 15 minutes, and then the cells were collected by centrifugation. The obtained cells were suspended in 5 mL of the fresh LB medium containing 1% citric acid, and the suspension was left at 30° C. for 30 minutes. Then, the cells were washed twice with the LB medium, inoculated on the LB agar medium (containing 40 mg/L of chloramphenicol), and cultured at 37° C. for 18 hours. In such a manner as described above, WC196ΔcadAΔldcC ydgT::cat strain was obtained, in which the ydgT gene was replaced with the attL-cat-attR gene.

Competent cells of the WC196ΔcadAΔldcC ydgT::cat strain obtained above were prepared in a conventional manner, transformed with the helper plasmid pMW-intxis-ts (WO2005/010175), and cultured at 30° C. for 24 hours on the LB agar medium containing 100 mg/L of ampicillin to select an ampicillin-resistant strain. Then, to remove the pMW-intxis-ts plasmid, the ampicillin-resistant strain was inoculated on the LB agar medium and cultured at 37° C. for 18 hours. Ampicillin resistance and chloramphenicol resistance of the obtained colonies were examined to obtain a ydgT gene-disrupted strain in which attL-cat-attR and pMW-intxis-ts had been eliminated, WC196ΔcadAΔldcCΔydgT. Disruption of the ydgT gene on the chromosome was confirmed by PCR using the synthetic DNAs shown in SEQ ID NOS: 7 and 8 as primers.

Into the obtained WC196ΔcadAΔldcCΔydgT strain and the parent strain thereof, WC196ΔcadAΔldcC strain, pCABD2 (WO95/16042) was introduced to obtain WC196ΔcadAΔldcCΔydgT/pCABD2 strain and WC196ΔcadAΔldcC/pCABD2 strain. Each of these two strains was inoculated on the LB agar medium (containing 100 mg/L of streptomycin), and cultured at 37° C. for 24 hours. The grown cells were scraped up, and suspended in L-lysine production medium A [60 g/L of glycerol, 16 g/L of (NH₄)₂SO₄, 1.0 g/L of KH₂PO₄, 1.0 g/L of MgSO₄.7H₂O, 0.01 g/L of FeSO₄.7H₂O, 0.01 g/L of MnSO₄.7H₂O, 2.0 g/L of yeast extract, and 30 g/L of CaCO₃ (Wako Pure Chemical Industries)] containing 100 mg/L of streptomycin.

The obtained cell suspension was inoculated into 20 mL of the medium A at a specific turbidity OD660 of 0.1, and culture was performed at 37° C. The culture broth was repeatedly sampled in a volume of 0.5 mL during the culture, and specific turbidity of the culture broth (OD660), L-lysine concentration in the culture broth, and glycerol concentration in the culture broth were measured for each sample. In order to measure OD660, the culture broth was diluted 51 times with 0.1 M HCl, and specific turbidity at 660 nm was measured with a spectrophotometer DU-800 (Beckman Coulter, USA). The L-lysine concentration in the culture broth was measured for the culture broth diluted 20 times with water by using Biotech Analyzer AS310 (Sakura Seiki, Japan). The glycerol concentration in the culture broth was measured for the culture broth diluted 20 times or 40 times with water by using a multifunctional biosensor BF-5 (Oji Scientific Instruments). At each culture time, the measurement was performed for four samples, except that the measurement was performed for nine samples immediately after the start of the culture and at 48 hours after the start of the culture.

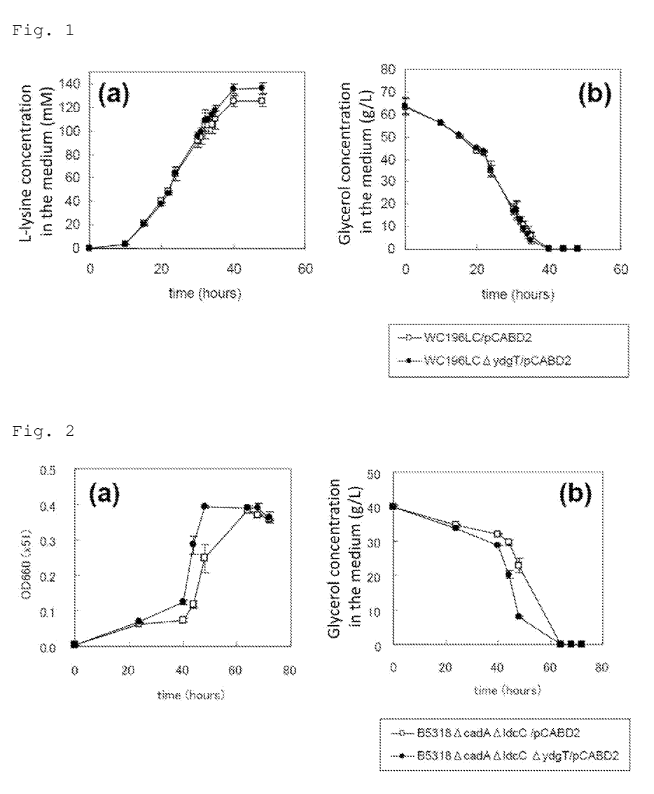

As a result, the L-lysine accumulation in the culture broth increased from 125.3±5.0 mM to 136.3±4.5 mM due to the deletion of the ydgT gene (FIG. 1). Furthermore, L-lysine yield based on glycerol was improved from 34.9±1.5(%) to 37.5±1.5(%).

### Example 3

### L-Lysine Production with Escherichia coli L-Lysine-Producing Bacterium Deficient in ydgT Gene (2)

As the L-lysine-producing bacterium, the Escherichia coli VKPM B-5318 strain (European Patent No. 0593792, henceforth referred to as “B5318 strain”), in which the cadA gene and the ldcC gene were disrupted, was used.

The cadA gene and the ldcC gene coding for lysine decarboxylase on the chromosome of the B5318 strain were disrupted in the same manner as that used for disruption of the ydgT gene in Example 1.

Specifically, disruption was performed as follows. PCR was performed by using the plasmid pMW118-attL-cat-attR as a template DNA, and synthetic oligo-DNAs shown in SEQ ID NOS: 3 and 4 or SEQ ID NOS: 5 and 6, respectively. After PCR, the template DNA was fully digested with the restriction enzyme DpnI, and the obtained PCR product was purified with Wizard® SV Gel and PCR Clean-Up System (Promega). Each PCR product was introduced into the Escherichia coli W3110 strain containing the plasmid pKD46 having temperature sensitive replication ability by electroporation. Competent cells for the electroporation were prepared in the same manner as that of Example 1. The PCR product (about 100 ng) was added to 300 μL of the competent cells, and electroporation was performed. The cells subjected to electroporation were cultured at 37° C. for 1 hour in 1 mL of the LB medium, and then inoculated on the LB agar medium (containing 40 mg/L of chloramphenicol). The cells were cultured at 37° C. for 18 hours to obtain a chloramphenicol resistant strain, and thereby obtain a W3110 strain in which the cadA gene or the ldcC gene on the chromosome was replaced with the attL-cat-attR gene.

Then, in order to successively disrupt the cadA gene and ldcC gene on the chromosome of the B5318 strain by P1 transduction using each of those gene-disrupted strains, P1 phages in which the chromosome of each disrupted strain was incorporated were prepared. First, the W3110 strain in which the cadA gene was disrupted was cultured at 37° C. for 18 hours in 1 mL of the LB medium. The obtained culture broth (50 μl) was added to the LB medium (4 mL), and culture was performed at 37° C. for 1 hour. The cells were collected from the obtained culture broth, and suspended in fresh LB medium (1 mL, containing 5 mM calcium chloride), P1 phage was added to the cell suspension, and the suspension was left at 37° C. for 1 hour. R-Top Agar (2.5 mL) was overlaid on the cell suspension infected with the P1 phage, the cell suspension was inoculated on the LB agar medium, and culture was performed at 37° C. for 14 hours. Then, 2 mL of the LB medium was added on the plate, the P1 phage and the cells were collected together with R-Top Agar, 30 μL of chloroform was added to them, and the supernatant was collected by centrifugation. After the addition of chloroform and collection of supernatant were repeated 3 times, the supernatant was filtered with a filter having a pore size of 0.2 μm to obtain a P1 phage solution for cadA gene disruption. Furthermore, a P1 phage solution for ldcC gene disruption was obtained in the same manner by using the W3110 strain in which the ldcC gene was disrupted.

Then, the cadA gene on the chromosome of the B5318 strain was disrupted by using the P1 phage for cadA gene disruption, specifically, as follows. The B5318 strain was cultured at 37° C. for 18 hours in 1 mL of the LB medium. The obtained culture broth (50 μL) was added to the LB medium (4 mL), and culture was performed at 37° C. for 1 hour. The obtained cells were collected, and suspended in fresh LB medium (1 mL, containing 5 mM calcium chloride), P1 phage was added to the cell suspension, and the suspension was left at room temperature for 15 minutes. To the suspension, 5 mL of the LB medium containing 1% citric acid was added, the mixture was left at 30° C. for 15 minutes, and then the cells were collected by centrifugation. The obtained cells were suspended in 5 mL of fresh LB medium containing 1% citric acid, and the suspension was left at 30° C. for 30 minutes. Then, the cells were washed twice with the LB medium, inoculated on the LB agar medium (containing 40 mg/L of chloramphenicol), and cultured at 37° C. for 18 hours to obtain B5318 cadA::cat strain, in which the cadA gene was replaced with the attL-cat-attR gene.

Competent cells of the B5318 cadA::cat strain obtained above were prepared in a conventional manner, transformed with the helper plasmid pMW-intxis-ts, and cultured at 30° C. for 24 hours on the LB agar medium containing 100 mg/L of ampicillin to select an ampicillin-resistant strain. Then, to remove the pMW-intxis-ts plasmid, the ampicillin-resistant strain was inoculated on the LB agar medium and cultured at 37° C. for 18 hours. Ampicillin resistance and chloramphenicol resistance of the obtained colonies were examined to obtain a cadA gene-disrupted strain, B5318ΔcadA strain, in which attL-cat-attR and pMW-intxis-ts had been eliminated. Disruption of the cadA gene on the chromosome was confirmed by PCR using the synthetic DNAs shown in SEQ ID NOS: 9 and 10 as primers.

Then, by using the P1 phage for ldcC gene disruption, the ldcC gene of the B5318ΔcadA strain was disrupted, and attL-cat-attR and pMW-intxis-ts were further eliminated in the same manner as that described above to obtain B5318ΔcadAΔldcC strain. From this strain, by using the P1 phage for ydgT gene disruption, the ydgT gene of this strain was disrupted, and attL-cat-attR and pMW-intxis-ts were further eliminated to obtain B5318ΔcadAΔldcCΔydgT strain. Disruption of the ldcC gene on the chromosome was confirmed by PCR using the synthetic DNAs shown in SEQ ID NOS: 11 and 12 as primers. Furthermore, disruption of the ydgT gene on the chromosome was confirmed by PCR using the synthetic DNAs shown in SEQ ID NOS: 8 and 9 as primers.

pCABD2 was introduced into the B5318ΔcadAΔldcCΔydgT strain and the B5318ΔcadAΔldcC strain to obtain B5318ΔcadAΔldcCΔydgT/pCABD2 strain and B5318ΔcadAΔldcC/pCABD2 strain.

Each of these two strains was inoculated on the LB agar medium (containing 100 mg/L of streptomycin), and cultured at 37° C. for 24 hours. The grown cells were scraped up, and suspended in L-lysine production medium B [40 g/L of glycerol, 16 g/L of (NH₄)₂SO₄, 1.0 g/L of KH₂PO₄, 1.0 g/L of MgSO₄.7H₂O, 0.01 g/L of FeSO₄.7H₂O, 0.01 g/L of MnSO₄.7H₂O, 2.0 g/L of yeast extract, and 30 g/L of CaCO₃ (Wako Pure Chemical Industries)] containing 100 mg/L of streptomycin. The obtained cell suspension was inoculated into 20 mL of the medium B at a specific turbidity OD660 of 0.1, and culture was performed at 37° C. Culture was performed with three samples for each strain, and specific turbidity of the culture broth (OD660), L-lysine concentration in the culture broth, and glycerol concentration in the culture broth were measured for each sample.

As a result, improvement in the proliferation rate and glycerol-assimilating ability due to the disruption of the ydgT gene was observed (FIG. 2).

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

1. A method for producing an L-amino acid comprising culturing a bacterium belonging to γ-Proteobacteria and having an ability to produce the L-amino acid in a medium containing glycerol as a carbon source to produce and accumulate the L-amino acid in the medium, and collecting the L-amino acid from the medium, wherein the bacterium is modified so that the activity of the Cnu protein is reduced as compared to a non-modified bacterium.

2. The method according to claim 1, wherein the activity of the Cnu protein is reduced by disrupting the ydgT gene coding for the Cnu protein on the chromosome, or by reducing expression amount of the gene.

3. The method according to claim 1, wherein the Cnu protein is a protein selected from the group consisting of:
(A) a protein having the amino acid sequence shown in SEQ ID NO: 14,
(B) a protein having the amino acid sequence shown in SEQ ID NO: 14, but including substitution, deletion, insertion or addition of one or several amino acid residues, reduction of which activity in the bacterium results in improvement in the ability to produce the L-amino acid.

4. The method according to claim 2, wherein the ydgT gene is a DNA selected from the group consisting of:
(a) a DNA comprising the nucleotide sequence of SEQ ID NO: 13,
(b) a DNA hybridizable with a nucleotide sequence complementary to the nucleotide sequence of SEQ ID NO: 13 or a probe that can be prepared from the nucleotide sequence under stringent conditions, and coding for a protein, reduction of which activity in the bacterium results in improvement in the ability to produce the L-amino acid.

5. The method according to claim 1, wherein the L-amino acid is an aspartic acid type amino acid.

6. The method according to claim 5, wherein the L-amino acid is L-lysine.

7. The method according to claim 1, wherein the bacterium is a bacterium belonging to the family Enterobacteriaceae.

8. The method according to claim 7, wherein the bacterium is an Escherichia bacterium.

9. The method according to claim 8, wherein the bacterium is Escherichia coli.


SEQUENCE LISTING (14 sequences)

SEQ ID NO: 1 - DNA, 70 bp, Artificial
  primer
atacctacccctttggtattaattattttatggacctttttgaagcctgcttttttatactaagttggca

SEQ ID NO: 2 - DNA, 70 bp, Artificial
  primer
ggcgttattataaggcttttataaccgatcgctactttctcgctcaagttagtataaaaaagctgaacga

SEQ ID NO: 3 - DNA, 70 bp, Artificial
  primer
catgtgttgggaggggccttttttacctggagatatgacttgaagcctgcttttttatactaagttggca

SEQ ID NO: 4 - DNA, 70 bp, Artificial
  primer
aaaaagggaagtggcaagccacttcccttgtacgagctaacgctcaagttagtataaaaaagctgaacga

SEQ ID NO: 5 - DNA, 70 bp, Artificial
  primer
agcaggctatgattaaggaaggattttccaggaggaacactgaagcctgcttttttatactaagttggca

SEQ ID NO: 6 - DNA, 70 bp, Artificial
  primer
gttaacagcacgttactcgcccggaagccgctctggcaagcgctcaagttagtataaaaaagctgaacga

SEQ ID NO: 7 - DNA, 20 bp, Artificial
  primer
ttgtaataacacaagcgttc

SEQ ID NO: 8 - DNA, 19 bp, Artificial
  primer
ttgtggtggtcataggttc

SEQ ID NO: 9 - DNA, 20 bp, Artificial
  primer
accgcgtctaacgcacatta

SEQ ID NO: 10 - DNA, 20 bp, Artificial
  primer
gagggtgttttcatgtgttc

SEQ ID NO: 11 - DNA, 20 bp, Artificial
  primer
gcaggctatgattaaggaag

SEQ ID NO: 12 - DNA, 20 bp, Artificial
  primer
atcgcaatatggtgaacctg

SEQ ID NO: 13 - DNA, 216 bp, Escherichia coli
  CDS (1)..(216)
atgactgttcaggactacttattaaaatttcgcaaaatcagttcactcgaaagtctggaaaaactctacgaccatcttaattacaccctgacggacgatcaggaactgatcaatatgtatcgtgctgccgatcaccgtcgcgcagagctggtttctggcgggcgtttgtttgacctcggccaggtaccgaagtccgtctggcactatgtccaataa

SEQ ID NO: 14 - protein, 71 aa, Escherichia coli
MTVQDYLLKFRKISSLESLEKLYDHLNYTLTDDQELINMYRAADHRRAELVSGGRLFDLGQVPKSVWHYVQ